(19) United States
(12) Patent Application Publication
(10) Pub. No.: US 2017/0340519 A9
(43) Pub. Date: Nov. 30, 2017

(54) DUAL-CHAMBER PACK FOR EXTENDED RELEASE SUSPENSION COMPOSITIONS

(71) Applicant: SUN PHARMACEUTICAL INDUSTRIES LIMITED, Mumbai (IN)

(72) Inventors: Rahul BHARGAVA, New Delhi (IN); Bhupesh Kumar MITTAL, Alwar (IN); Kalaiselvan RAMARAJU, Trichirapalli (IN); Ashish KUMAR, Jhajjar (IN)

(21) Appl. No.: 15/352,993

(22) Filed: Nov. 16, 2016

Related U.S. Application Data
(62) Division of application No. 15/144,058, filed on May 2, 2016.
Application No. 15/144,058 is a continuation-in-part of application No. 15/133,826, filed on Apr. 20, 2016.
Application No. 15/133,826 is a continuation of application No. PCT/IB2015/053209, filed on May 1, 2015.
Application No. 15/144,058 is a continuation-in-part of application No. PCT/IB2015/055780, filed on Jul. 30, 2015.

(30) Foreign Application Priority Data
May 1, 2014 (IN) 1183/DEL/2014
Jul. 30, 2014 (IN) 2149/DEL/2014

(51) Int. Cl. A61J 1/20; A61K 47/36; A61K 47/26; A61K 31/155; A61J 1/14; A61K 9/50; A61K 9/10; A61K 9/00; A61K 47/38; A61K 47/14

(52) U.S. Cl. CPC A61J 1/2093; A61K 47/38; A61K 47/36; A61K 47/26; A61K 47/14; A61J 1/1418; A61K 9/5042; A61K 9/5015; A61K 9/10; A61K 9/0095; A61K 31/155

(57) ABSTRACT

The present invention relates to a dual-chamber pack comprising a first chamber prefilled with a suspension base and a second chamber prefilled with a powder for suspension comprising an active ingredient, wherein upon activation of the dual-chamber pack, the contents of both the chambers are mixed to form an extended release suspension composition which is characterized by having no substantial change in the in-vitro dissolution release profile of the active ingredient upon storage for at least seven days.

### FIELD OF THE INVENTION

The present invention relates to a dual-chamber pack comprising a first chamber prefilled with a suspension base and a second chamber prefilled with a powder for suspension comprising an active ingredient, wherein upon activation of the dual-chamber pack, the contents of both the chambers are mixed to form an extended release suspension composition which is characterized by having no substantial change in the in-vitro dissolution release profile of the active ingredient upon storage for at least seven days.

### BACKGROUND OF THE INVENTION

Extended release solid compositions are preferred dosage forms over immediate release solid compositions, especially for active ingredients showing fluctuations in the plasma concentration and for active ingredients having short half-lives. Extended release solid compositions can be in the form of tablets or capsules, wherein the release of the active ingredient is controlled by using a reservoir or a matrix system. However, extended release solid compositions suffer from certain drawbacks such as difficulty in swallowing, particularly for certain groups of patients, e.g., pediatrics and geriatrics, resulting in poor patient compliance. Further, high doses of active ingredients lead to large-sized compositions which aggravates this problem. Also, there remains a tendency to divide extended release solid compositions such as tablets into small pieces in order to facilitate administration, which may ultimately lead to inaccurate dosing and/or dose dumping. In view of all this, extended release liquid compositions provide the best alternative over extended release solid compositions. Extended release liquid compositions are easy to administer, thereby leading to enhanced patient compliance. Additionally, extended release liquid compositions provide a unique advantage of having a flexible dosing regimen.

Extended release liquid compositions are conventionally administered as powder for suspensions which are to be reconstituted by the end users at the time of administration using household pre-boiled and cooled water. Alternatively, the diluent or purified water is supplied separately along with the bottle having the extended release powder for suspension. These conventional packs lack patient compliance and may lead to contamination due to improper quality of water. Further, there remains a possibility of dosing errors if the diluent or water is not added to the marked level.

U.S. Pat. No. 3,156,369; U.S. Pat. No. 3,603,469; U.S. Pat. No. 3,840,136; and U.S. Pat. No. 4,982,875 disclose the use of dual-chamber packs for separately storing two compositions in two compartments which can be admixed at the time of use. The two compartments are separated by a breakable membrane which is ruptured by the depression of a plunger so that the one composition gets released into another and is mixed. However, there remains a possibility that the membrane fragments may get detached and fall into the final product. This may lead to undesirable contamination and can pose serious health hazards. Furthermore, the dual-chamber packs disclosed in the prior art have a limited capacity for the compartments which may not be suitable for high-dose drugs or for drugs which require chronic administration. Also, the liquid composition may get permeated into the solid composition across the membrane during storage which can lead to the agglomeration of the solid composition. This may result in poor flow of the solid composition, thus affecting the content uniformity of the final product. Also, the liquid composition on permeation can affect the stability of moisture-sensitive active ingredients.

The present invention provides a patient compliant dual-chamber pack with a significant improvement over the prior art and which fulfills the unmet need of incorporating variety of active ingredients. The present dual-chamber pack can be suitable for any class of active ingredients including the high-dose active ingredients, active ingredients requiring chronic administration, and/or moisture-sensitive active ingredients. Further, the plunger used in the pack of the instant invention is designed in a way such that the breakable membrane remains adhered to the plug at the time of activation and membrane fragments do not fall into the final product. During activation, the pack ensures that the final product remains safe for the use of patients. The pack also ensures that the solid composition is completely released into the liquid composition thereby maintaining the content uniformity of the final product. Further, the pack also ensures that there is no permeation of moisture into the chamber having solid composition comprising the active ingredient, and the stability of the active ingredient remains unaffected during storage.

Apart from storage, there remains some of the complexities involved in formulating such reconstituted extended release powder for suspension compositions. Upon reconstitution, the important prerequisite of these compositions is to provide the desired extended release of the active ingredient throughout its shelf life, as irregular release may lead to sub-therapeutic or toxic effects. Once reconstituted, the key hurdle remains to overcome the leaching of the active ingredient from the coated cores into a suspension base during storage. The objective for a scientist remains to develop a formulation such that the release of the active ingredient into the suspension base during storage is avoided, and only when the suspension enters the gastrointestinal tract the release is allowed.

The present invention offers the reconstituted suspension compositions which provide the desired extended release of the active ingredient throughout the shelf life of the compositions. In the present invention, the suspension base prevents the leaching of the active ingredient from the coated cores and thus ensures substantially similar in-vitro dissolution release profile of the active ingredient throughout the shelf life of the compositions. This consistent in-vitro release then ensures a steady plasma concentration with no fluctuations throughout the shelf life of the compositions.

The present invention thus provides a novel patient-compliant dual-chamber pack prefilled with solid and liquid compositions in two chambers, which upon mixing forms a unique composition providing the desired extended release of the active ingredient throughout the shelf life of the composition. The compositions prefilled in the dual-chamber pack remain stable during the storage.

### SUMMARY OF THE INVENTION

The present invention relates to a dual-chamber pack comprising a first chamber prefilled with a suspension base and a second chamber prefilled with a powder for suspension comprising an active ingredient, wherein upon activation of the dual-chamber pack, the contents of both the chambers are mixed to form an extended release suspension composition which is characterized by having no substantial change in the in-vitro dissolution release profile of the active ingredient upon storage for at least seven days. The pack allows the end-users ease of dispensing with only a few simple steps required for reconstitution. The pack is suitable from low to high dose active ingredients, active ingredients required for chronic administration as well as moisture-sensitive active ingredients. The pack ensures that the powder for suspension falls completely into the suspension base thereby maintaining the content uniformity. The pack also ensures that final product remains free of any contamination from the pack components and is safe to the end-users. Further, the pack ensures the stability of the active ingredient during storage.

### BRIEF DESCRIPTION OF THE DRAWINGS

FIG. 1: Schematic diagram of the components of a dual-chamber pack with a powder for suspension prefilled in the plunger

Overcap—1, Tamper evident band—2, Plunger—3, Plug—4, Breakable polymeric membrane—5, Opening of the container—6, Container—7

FIG. 2: Schematic diagram of the components of a dual-chamber pack with a powder for suspension prefilled in the reservoir

Reservoir—1, Plunger—2, Plug—3, Breakable polymeric membrane—4, Biphasic connector—5, Tamper evident band—6, Opening of the container—7, Container—8, Cap—9

FIG. 3: Schematic diagram for the biphasic connector—top view and front view

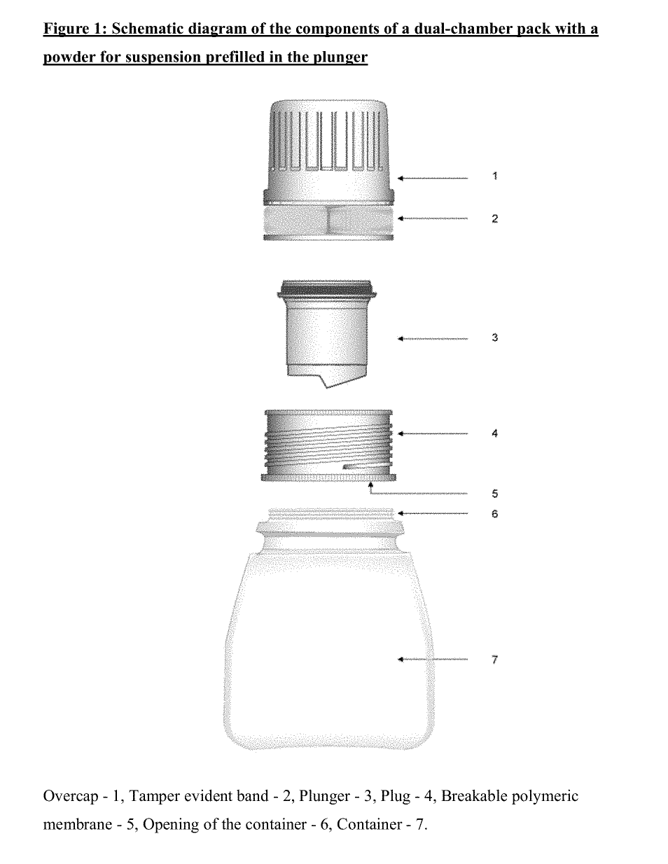

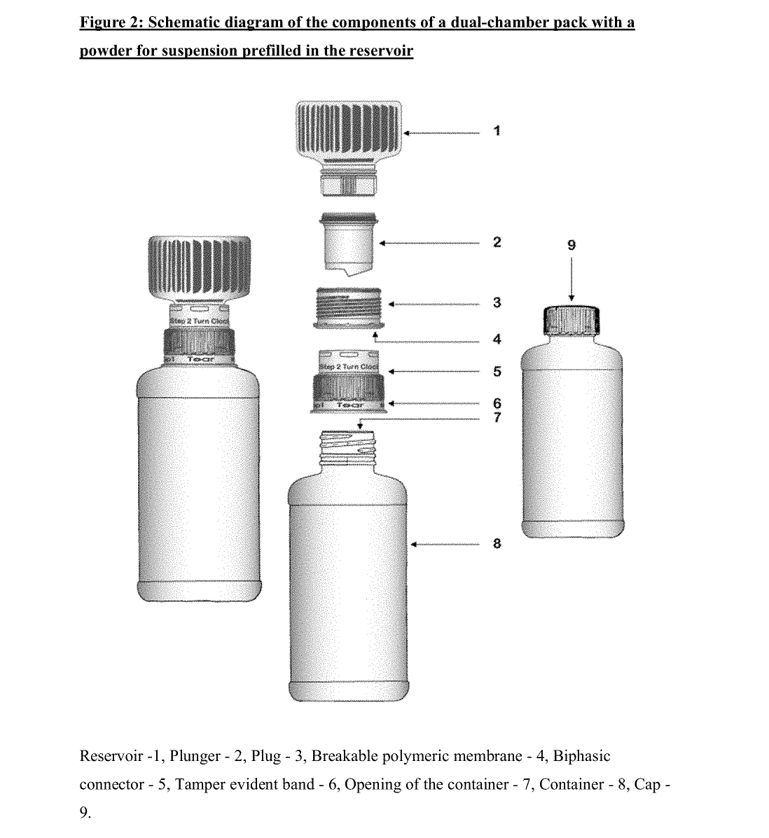

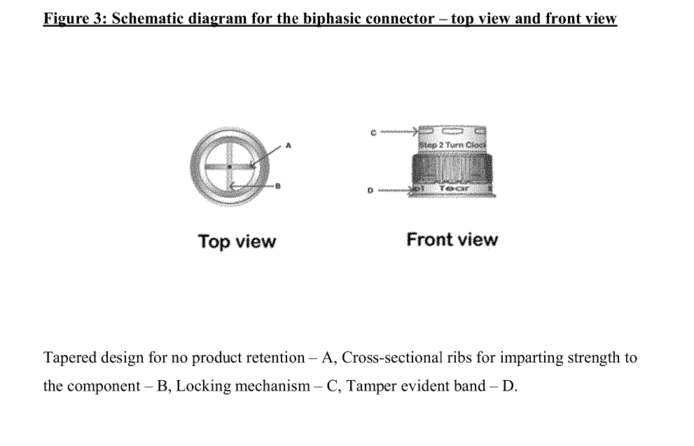

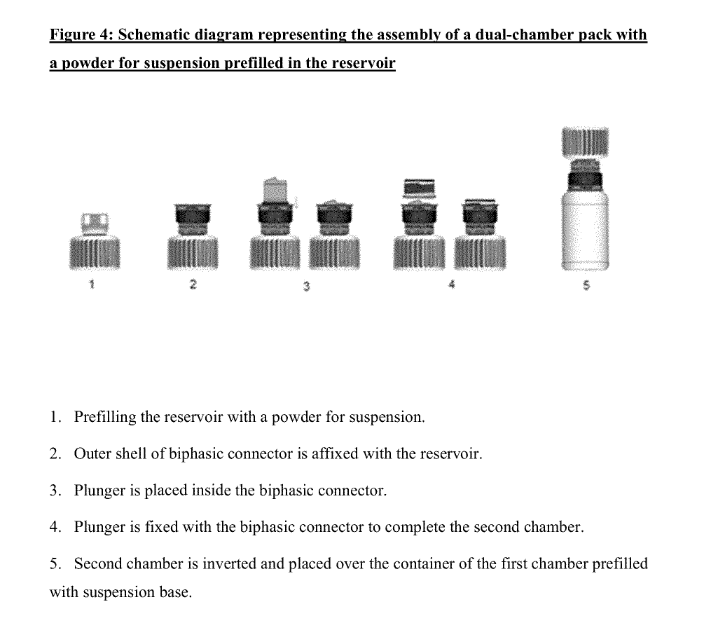

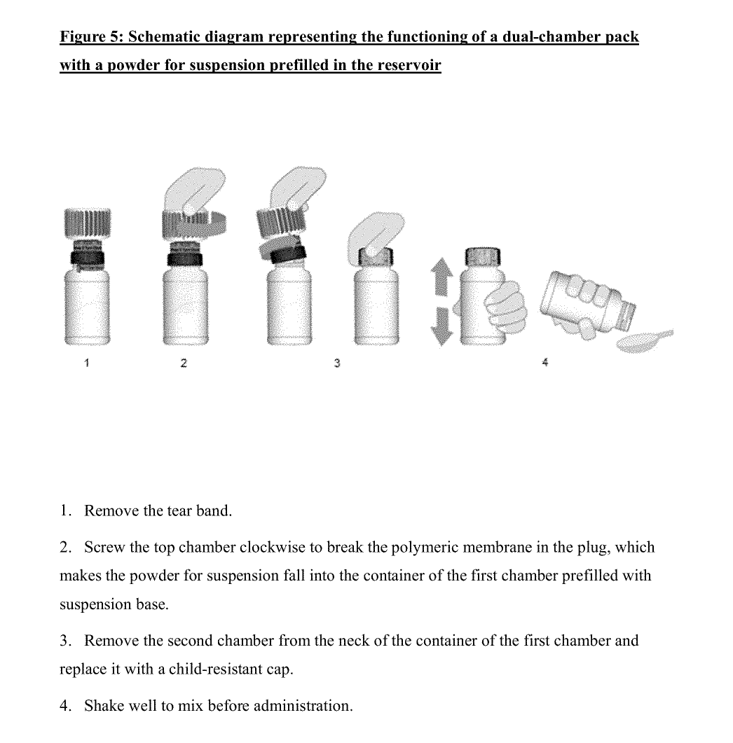

Tapered design for no product retention—A, Cross-sectional ribs for imparting strength to the component—B, Locking mechanism—C, Tamper evident band—D

FIG. 4: Schematic diagram representing the assembly of a dual-chamber pack with a powder for suspension prefilled in the reservoir
1. Prefilling the reservoir with a powder for suspension.
2. Outer shell of biphasic connector is affixed with the reservoir.
3. Plunger is placed inside the biphasic connector.
4. Plunger is fixed with the biphasic connector to complete the second chamber.
5. Second chamber is inverted and placed over the container of the first chamber prefilled with suspension base.

FIG. 5: Schematic diagram representing the functioning of a dual-chamber pack with a powder for suspension prefilled in the reservoir
1. Remove the tear band.
2. Screw the top chamber clockwise to break the polymeric membrane in the plug, which makes the powder for suspension fall into the container of the first chamber prefilled with suspension base.
3. Remove the second chamber from the neck of the container of the first chamber and replace it with a child-resistant cap.
4. Shake well to mix before administration.

### DETAILED DESCRIPTION OF THE INVENTION

A first aspect of the invention provides a dual-chamber pack comprising;
(a) a first chamber prefilled with a suspension base; and
(b) a second chamber prefilled with a powder for suspension comprising an active ingredient;
   wherein upon activation of the dual-chamber pack, the contents of both the chambers are mixed to form an extended release suspension composition which is characterized by having no substantial change in the in-vitro dissolution release profile of the active ingredient upon storage for at least seven days.

According to one embodiment of the above aspect, the powder for suspension prefilled in the second chamber is present in a volume ranging from about 0.5 cc to about 500 cc.

According to another embodiment of the above aspect, the first chamber comprises of a container and the second chamber comprises of an overcap, a plunger, and a plug with a breakable polymeric membrane. The plunger is prefilled with the powder for suspension in a volume ranging from about 0.5 cc to about 30 cc.

According to another embodiment of the above aspect, the first chamber comprises of a container and the second chamber comprises of a reservoir, a biphasic connector, a plunger, and a plug with a breakable polymeric membrane. The reservoir is prefilled with the powder for suspension in a volume greater than about 30 cc. In particular, the reservoir is prefilled with the powder for suspension in a volume ranging from about 30 cc to about 500 cc.

According to another embodiment of the above aspect, the biphasic connector of the second chamber connects the reservoir to the container of the first chamber.

According to another embodiment of the above aspect, the plunger ensures the breakable polymeric membrane remains attached to the plug during activation.

According to another embodiment of the above aspect, the plunger comprise of one or more sharp projections with an essential continuous blunt area. In a preferred embodiment, the plunger comprise of one sharp projection with an essential continuous blunt area. The plunger can further have one or more grooves. The body of the plunger can be in the form of a cylinder or a funnel.

According to another embodiment of the above aspect, the plug is made up of a polymeric material selected from the group comprising polyolefin, polyethylene, polypropylene, polyvinyl chloride, cyclic olefin polymer, cyclic olefin co-polymer, polyethylene terephthalate, polyethylene terephthalate-G, polypropylene, and polycarbonate. In a preferred embodiment, the plug is made up of polyethylene.

According to another embodiment of the above aspect, the plug additionally includes one or more moisture barrier additives.

According to another embodiment of the above aspect, the moisture barrier additives are selected from the plastic additive group comprising of monomers and co-polymers that get activated through polymerization process to form an effective organic chemical.

According to another embodiment of the above aspect, the moisture barrier additives improve the moisture barrier properties by up to 50%. In particular, the moisture barrier additives improve the moisture barrier properties by up to 30%.

According to another embodiment of the above aspect, the plug with the breakable polymeric membrane prevents moisture permeation from the first chamber into the second chamber.

According to another embodiment of the above aspect, the extended release suspension composition is a stable composition.

A second aspect of the present invention provides a dual-chamber pack comprising:

a) a first chamber in the form of a container (7) prefilled with a suspension base and provided with an opening (6) at an upper end;

b) a second chamber comprising:
(i) a overcap (1) optionally having a tamper evident band (2) fitted into a plunger (3);
(ii) the plunger (3) adapted to fit into a plug (4), having a top flat surface, prefilled with a powder for suspension comprising an active ingredient;
(iii) the plug (4), with a breakable polymeric membrane (5), adapted to fit into the opening (6) from a lower end and into the overcap (1) from the upper end; and
   wherein the overcap (1) has a means to exert pressure onto the plunger (3) so as to partially rupture the breakable polymeric membrane (5) of the plug and deliver the powder for suspension into the suspension base of the container (7); and wherein the powder for suspension is mixed with the suspension base to form an extended release suspension composition which is characterized by having no substantial change in the in-vitro dissolution release profile of the active ingredient upon storage for at least seven days.

According to one embodiment of the above aspect, the plunger is prefilled with the powder for suspension in a volume ranging from about 0.5 cc to about 30 cc.

According to another embodiment of the above aspect, the plunger may be opened at both the ends. In this case, the plunger is fitted into the overcap first, and then the powder for suspension is prefilled into the plunger which is then fitted with a plug.

According to another embodiment of the above aspect, the plunger comprise of one or more sharp projections with an essential continuous blunt area. In a preferred embodiment, the plunger comprise of one sharp projection with an essential continuous blunt area.

The overcap exerts pressure onto the plunger when it is screwed during activation of the dual-chamber pack.

A third aspect of the present invention provides a dual-chamber pack comprising:

a) a first chamber in the form of a container (8) prefilled with a suspension base provided with an opening (7) at an upper end;

b) a second chamber comprising:
(i) a reservoir (1) adapted to fit into a plunger (2) prefilled with a powder for suspension comprising an active ingredient; the plunger (2) is further adapted to fit into a plug (3) having a top flat surface,
(ii) the plug (3), with a breakable polymeric membrane (4), adapted to fit into the biphasic connector (5) optionally having a tamper evident band (6) which is further connected from the lower end to the opening (7) of the container (8);
   wherein the reservoir (1) at the top of the second chamber has a means to exert pressure onto the plunger (2) so as to partially rupture the breakable polymeric membrane (4) of the plug and deliver the powder for suspension into the suspension base of the container (8); the second chamber is replaced with a cap (9), and wherein the powder for suspension is mixed with the suspension base to form an extended release suspension composition which is characterized by having no substantial change in the in-vitro dissolution release profile of the active ingredient upon storage for at least seven days.

According to one embodiment of the above aspect, the reservoir is prefilled with the powder for suspension in a volume greater than about 30 cc, particularly in a range from about 30 cc to about 500 cc.

According to another embodiment of the above aspect, the plunger comprise of one or more sharp projections, wherein the plunger essentially has a continuous blunt area. In a preferred embodiment, the plunger comprise of one sharp projection with a continuous blunt area. The body of the plunger can be in the form of a cylinder or a funnel. The funnel shaped plunger further helps to increase the capacity to incorporate high dose drugs.

According to another embodiment of the above aspect, the plunger is opened at both the ends.

According to another embodiment of the above aspect, the cap is a conventional cap or a child-resistant cap.

According to another embodiment of the above aspect, the biphasic connector has a tamper evident band on the side connected to the container of the first chamber and grooves on another side for locking with the reservoir of the second chamber.

According to another embodiment of the above aspect, the reservoir exerts pressure onto the plunger when it is screwed during activation of the dual-chamber pack.

A fourth aspect of the present invention provides a method of providing an extended release suspension composition stored in a dual-chamber pack, comprising the steps of:

(a) providing a first chamber comprising a container (7), a second chamber comprising an overcap (1), a plunger (3), a plug (4) with a breakable polymeric membrane (5);

(b) prefilling the container (7) of the first chamber with a suspension base;

(c) prefilling the plunger (3) of the second chamber with a powder for suspension comprising an active ingredient;

(d) fixing the plunger (3) into the plug (4) and mounting the plug on an opening (6) of the container (7) of the first chamber;

(e) activating the dual-chamber pack by screwing the overcap (1) so that the plunger (3) partially ruptures breakable polymeric membrane (5) of the plug (4); and

(f) shaking the container (7) to allow the mixing of the powder for suspension with the suspension base to obtain the extended release suspension composition which is characterized by having no substantial change in the in-vitro dissolution release profile of the active ingredient upon storage for at least seven days.

According to one embodiment of above aspect, the plunger is prefilled with the powder for suspension in a volume ranging from about 0.5 cc to about 30 cc.

According to another embodiment of above aspect, the plunger may be open at both the ends. In this case, the plunger is fitted into the overcap first, and then the powder for suspension is prefilled into the plunger which is then fitted with a plug. Alternatively, the overcap may be prefitted with the plunger.

The overcap may have a tamper-evident band which is to be removed first to start the activation process.

A fifth aspect of the present invention provides a method of providing an extended release suspension composition stored in a dual-chamber pack, comprising the steps of:

(a) providing a first chamber comprising a container (8), a second chamber comprising a reservoir (1), a plunger (2), a plug (3) with a breakable polymeric membrane (4), and a biphasic connector (5);

(b) prefilling the container (8) of the first chamber with a suspension base to form a first chamber;

(c) prefilling a reservoir (1) of the second chamber with a powder for suspension comprising an active ingredient;

(d) fixing the biphasic connector (5) into the reservoir (1);

(e) fixing the plunger (2) in the biphasic connector (5);

(f) mounting the plug (3) onto the plunger of the biphasic connector (5) to form the second chamber;

(g) mounting the second chamber onto the opening (7) of the container (8) of the first chamber;

(h) activating the dual-chamber pack by screwing the reservoir (1) of the second chamber so that the plunger partially ruptures the circumference of a breakable polymeric membrane; and

(i) removing the second chamber and replacing it with a cap (9);

(j) shaking the container (8) to allow the mixing of the powder for suspension with the suspension base to obtain the extended release suspension composition which is characterized by having no substantial change in the in-vitro dissolution release profile of the active ingredient upon storage for at least seven days.

According to one embodiment of the above aspect, the reservoir is prefilled with the powder for suspension in a volume greater than about 30 cc, particularly in a range from about 30 cc to about 500 cc.

According to another embodiment of above aspect, the biphasic connector has a tamper evident band on the side connected to the container of the first chamber and grooves on another side for locking with the reservoir of the second chamber. The tamper evident band is removed first to start the activation process.

According to another embodiment of the above aspects, the powder for suspension comprise of extended release coated cores of an active ingredient, optionally admixed with one or more pharmaceutically acceptable excipients. The powder for suspension may additionally have one or more osmogents, or one or more suspending agents. The core may comprise of a release-controlling agent in the form of a matrix with the active ingredient, which can be coated with a coating layer that remain insoluble in the suspension base during storage.

According to another embodiment of the above aspects, the extended release coated cores comprise a core comprising an active ingredient and a coating layer over said core comprising one or more release-controlling agents.

According to another embodiment of the above aspects, the core is in the form of a bead, a pellet, a granule, a spheroid, or the like.

According to another embodiment of the above aspects, the active ingredient is layered onto an inert particle to form the core.

Alternatively, the extended release coated cores comprise a core comprising an active ingredient in a complexed or an ion-exchange resin form and a coating layer over said core comprising one or more release-controlling agents.

According to another embodiment of above aspects, the release-controlling agent is selected from the group comprising a pH-dependent release-controlling agent, a pH-independent release-controlling agent, or mixtures thereof.

According to another embodiment of the above aspects, the extended release suspension composition is characterized by having an osmolality ratio of at least about 1.

The term “powder for suspension,” as used herein, refers to a solid composition comprising extended release coated cores of an active ingredient, optionally admixed with one or more osmogents, one or more suspending agents, and/or one or more pharmaceutically acceptable excipients. The plunger or reservoir of the second chamber of the present invention is prefilled with the powder for suspension.

The term “suspension base,” as used herein, refers to a medium which is used to suspend the coated cores of the active ingredient. The suspension base of the present invention comprises one or more suspending agents, one or more osmogents, and a pharmaceutically acceptable vehicle. It may further comprise one or more pharmaceutically acceptable excipients. The powder for suspension having coated cores of active ingredient may be reconstituted with the suspension base having suspending agents, osmogents, pharmaceutically acceptable excipients, and a pharmaceutically acceptable vehicle. Alternatively, suspending agents, osmogents, or other pharmaceutically acceptable excipients may be premixed with the coated cores which may be reconstituted with the pharmaceutically acceptable vehicle. The pharmaceutically acceptable vehicle may comprise of purified water or a mixture of purified water with one or more suitable organic solvents, in particular purified water. The container of the first chamber of the present invention is prefilled with a pre-formed suspension base or a pharmaceutically acceptable vehicle which forms the suspension base at the time of reconstitution. The suspension base generates a hypertonic condition such that there is no substantial change in the in-vitro dissolution release profile of the active ingredient upon storage of the reconstituted extended release suspension composition for at least seven days. The suspension base of the present invention has an osmolality of at least about 1 osmol/kg of the suspension base.

The term “activation,” as used herein means a process which reconstitutes the powder for suspension with the suspension base. The activation can be done by the end-users such as patients, pharmacists, or caregivers. The activation process starts by either screwing the overcap or the reservoir.

The term “extended release,” as used herein, refers to the release profile of the active ingredient over an extended period of time, e.g., over a period of 4, 6, 8, 12, 24 hours, or more.

The term “hypertonic condition,” as used herein, means the suspension base has higher solute concentration which helps to generate high osmotic pressure such that there is no significant leaching of active ingredient from the coated cores into the suspension base. In the present invention, the solutes are osmogents i.e., pharmaceutically acceptable inert water-soluble compounds that contribute towards generating hypertonic conditions in the suspension base. Alternatively, a saturated solution of the active ingredient present in the suspension base or the external phase may prevent the substantial leaching of the active ingredient from the extended release coated cores.

The term “osmolality ratio,” as used herein, means the ratio of the osmolality of the external phase to the osmolality of the internal phase. The external phase herein means the suspension base without the multiple extended release coated cores of the active ingredient. The internal phase herein means the extended release coated cores of the active ingredient. As the direct measurement of the osmolality of the internal phase i.e., coated cores is difficult, the osmolality of the internal phase herein, is represented as the osmolality of a solution which prevents significant leaching of the active ingredient from the coated cores into the solution. The leaching of the active ingredient from the extended release coated cores is determined by the difference in the osmolalities across the coating layer and the absence of any significant leaching from the extended release coated cores directs that the osmolality of the solution has become equal to the osmolality of the extended release coated cores. The osmolality ratio of the extended release suspension compositions of present invention is at least about 1.

The term “osmolality,” as used herein, is expressed as number of moles of any water-soluble compound per kg of a liquid phase. The liquid phase can be a suspension base or a solution. In the present invention, the osmolality may be measured according to known methods, such as using a vapor pressure osmometer, a colloid osmometer, or a freezing point depression osmometer such as Osmomat 030-D or Osmomat 3000, in particular by a freezing point depression osmometer.

The term “inert particle,” as used herein, refers to a particle made from a sugar sphere also known as a non-pareil seed, a microcrystalline cellulose sphere, a dibasic calcium phosphate bead, a mannitol bead, a silica bead, a tartaric acid pellet, a wax based pellet, and the like.

The term “substantial,” as used herein refers to any value which lies within the range as defined by a variation of up to ±15 from the average value.

The term “about” as used herein, refers to any value which lies within the range defined by a variation of up to ±10% of the value.

The term “significant leaching,” as used herein means more than 20% of the active ingredient is leached out from the extended release coated cores into the solution.

The term “stable,” as used herein, refers to chemical stability, wherein not more than 5% w/w of total related substances are formed on storage at 40° C. and 75% relative humidity (R.H.) or at 25° C. and 60% R.H. for a period of at least three months to the extent necessary for the sale and use of the composition.

The term “osmogent,” as used herein, refers to all pharmaceutically acceptable inert water-soluble compounds that can imbibe water and/or aqueous biological fluids. The osmogent can be present in the suspension base or in the powder for suspension or both. Suitable examples of osmogents or pharmaceutically acceptable inert water-soluble compounds are selected from the group comprising carbohydrates such as xylitol, mannitol, sorbitol, arabinose, ribose, xylose, glucose, fructose, mannose, galactose, sucrose, maltose, lactose, dextrose and raffinose; water-soluble salts of inorganic acids such as magnesium chloride, magnesium sulfate, potassium sulfate, lithium chloride, sodium chloride, potassium chloride, lithium hydrogen phosphate, sodium hydrogen phosphate, potassium hydrogen phosphate, lithium dihydrogen phosphate, sodium dihydrogen phosphate, potassium dihydrogen phosphate, and sodium phosphate tribasic; water-soluble salts of organic acids such as sodium acetate, potassium acetate, magnesium succinate, sodium benzoate, sodium citrate, and sodium ascorbate; water-soluble amino acids such as glycine, leucine, alanine, methionine; urea or its derivatives; propylene glycol; glycerin; polyethylene oxide; xanthan gum; hydroxypropylmethyl cellulose; and mixtures thereof. Particularly, the osmogents used in the present invention are xylitol, mannitol, glucose, lactose, sucrose, and sodium chloride.

Suitable suspending agents are selected from the group comprising cellulose derivatives such as co-processed spray dried forms of microcrystalline cellulose and carboxymethyl cellulose sodium, hydroxypropyl cellulose, hydroxyethyl cellulose, hydroxypropylmethyl cellulose, methylcellulose, carboxymethyl cellulose and its salts/derivatives, and microcrystalline cellulose; carbomers; gums such as locust bean gum, xanthan gum, tragacanth gum, arabinogalactan gum, agar gum, gellan gum, guar gum, apricot gum, karaya gum, sterculia gum, acacia gum, gum arabic, and carrageenan; pectin; dextran; gelatin; polyethylene glycols; polyvinyl compounds such as polyvinyl acetate, polyvinyl alcohol, and polyvinyl pyrrolidone; sugar alcohols such as xylitol and mannitol; colloidal silica; and mixtures thereof. Co-processed spray dried forms of microcrystalline cellulose and carboxymethyl cellulose sodium have been marketed under the trade names Avicel® RC-501, Avicel® RC-581, Avicel® RC-591, and Avicel® CL-611. The term “pharmaceutically acceptable excipients,” as used herein, refers to excipients that are routinely used in pharmaceutical compositions. The pharmaceutically acceptable excipients may comprise glidants, sweeteners, anti-caking agents, wetting agents, preservatives, buffering agents, flavoring agents, anti-oxidants, chelating agents, and combinations thereof.

The average diameter of the extended release coated cores ranges from about 10 μm to about 2000 μtm, particularly from about 50 μm to about 1000 μm, and more particularly from about 150 μm to about 500 μm. The finer sizes of the extended release coated cores help in avoiding grittiness in the mouth and are therefore more acceptable.

This dual-chamber pack can be used for active ingredients such as valacyclovir, metformin, azithromycin, cloxacillin, clarithromycin, erythromycin, amoxicillin alone or in combination with clavulanic acid, cefdinir, cefuroxime axetil, cefixime, cefadroxil, cefpodoxime, cefaclor, cefprozil, fluconazole, voriconazole, acarbose, miglitol, voglibose, repaglinide, nateglinide, glibenclamide, glimepride, glipizide, gliclazide, chloropropamide, tolbutamide, phenformin, alogliptin, sitagliptin, linagliptin, saxagliptin, rosiglitazone, pioglitazone, troglitazone, faraglitazar, englitazone, darglitazone, isaglitazone, zorglitazone, liraglutide, muraglitazar, peliglitazar, tesaglitazar, canagliflozin, dapagliflozin, remogliflozin, sergliflozin, verapamil, albuterol, salmeterol, acebutolol, sotalol, penicillamine, norfloxacin, ciprofloxacin, ofloxacin, levofloxacin, moxifloxacin, trovafloxacin, gatifloxacin, tetracycline, demeclocycline hydrochloride, losartan, irbesartan, eprosartan, valsartan, diltiazem, isosorbide mononitrate, ranolazine, propafenone, hydroxyurea, hydrocodone, delavirdine, pentosan polysulfate, abacavir, amantadine, acyclovir, ganciclovir, valganciclovir, saquinavir, indinavir, nelfinavir, lamivudine, didanosine, zidovudine, nabumetone, celecoxib, mefenamic acid, naproxen, propoxyphene, cimetidine, ranitidine, albendazole, mebendazole, thiobendazole, pyrazinamide, praziquantel, chlorpromazine, sumatriptan, bupropion, aminobenzoate, pyridostigmine bromide, potassium chloride, niacin, tocainide, quetiapine, fexofenadine, sertraline, chlorpheniramine, rifampin, methenamine, nefazodone, modafinil, metaxalone, morphine, sevelamer, lithium carbonate, flecainide acetate, simethicone, methyldopa, chlorthiazide, metyrosine, procainamide, entacapone, metoprolol, propanolol hydrochloride, chlorzoxazone, tolmetin, tramadol, bepridil, phenytoin, gabapentin, terbinafine, atorvastatin, doxepine, rifabutin, mesalamine, etidronate, nitrofurantoin, choline magnesium trisalicylate, theophylline, nizatidine, methocarbamol, mycophenolate mofetil, tolcapone, ticlopidine, capecitabine, orlistat, colsevelam, meperidine, hydroxychloroquine, guaifenesin, guanfacine, amiodarone, quinidine, atomoxetine, felbamate, pseudoephedrine, carisoprodol, venlafaxine, etodolac, chondroitin, lansoprazole, pantoprazole, esomeprazole, dexlansoprazole, dexmethylphenidate, methylphenidate, sodium oxybate, valproic acid or its salts, divalproex, topiramate, carbamazepine, oxcarbazepine, isotretinoin, oseltamivir, cholestyramine, nystatin, and a combination of artemether and lumefantrine.

The suspension base or the powder for suspension of the present invention may further include an immediate release component of the active ingredient to have a biphasic or pulsatile type of release. The immediate release component may be present in the form of a powder, a pellet, a bead, a spheroid, or a granule. Alternatively, the immediate release component may be present in the form of an immediate release coating over the extended release coated cores. The reconstituted extended release suspension composition of the present invention may comprise two or more different active ingredients with different type of release profiles or incompatible active ingredients.

The release-controlling agents used to form the extended release coating are selected from a group comprising a pH-dependent release-controlling agent, a pH-independent release-controlling agent, or mixtures thereof.

Suitable examples of pH-dependent release-controlling agents are selected from the group comprising acrylic copolymers such as methacrylic acid and methyl methacrylate copolymers, e.g., Eudragit® L 100 and Eudragit® S 100, methacrylic acid and ethyl acrylate copolymers, e.g., Eudragit® L 100-55 and Eudragit® L 30 D-55, dimethylaminoethyl methacrylate and butyl methacrylate and methyl methacrylate copolymers e.g., Eudragit® E 100, Eudragit® E PO, methyl acrylate and methacrylic acid and octyl acrylate copolymers, styrene and acrylic acid copolymers, butyl acrylate and styrene and acrylic acid copolymers, and ethylacrylate-methacrylic acid copolymer; cellulose acetate phthalate; cellulose acetate succinates; hydroxyalkyl cellulose phthalates such as hydroxypropylmethyl cellulose phthalate; hydroxyalkyl cellulose acetate succinates such as hydroxypropylmethyl cellulose acetate succinate; vinyl acetate phthalates; vinyl acetate succinate; cellulose acetate trimelliate; polyvinyl derivatives such as polyvinyl acetate phthalate, polyvinyl alcohol phthalate, polyvinyl butylate phthalate, and polyvinyl acetoacetal phthalate; zein; shellac; and mixtures thereof.

Suitable examples of pH-independent release-controlling agents are selected from the group comprising cellulosic polymers such as ethyl cellulose, methyl cellulose, hydroxyethyl cellulose, hydroxypropyl cellulose, hydroxyethylmethyl cellulose, hydroxypropylmethyl cellulose, and carboxy methylcellulose; acrylic copolymers such as methacrylic acid copolymers, e.g., Eudragit® RS, Eudragit® RL, Eudragit® NE 30 D; cellulose acetate; polyethylene derivatives e.g., polyethylene glycol and polyethylene oxide; polyvinyl alcohol; polyvinyl acetate; gums e.g., guar gum, locust bean gum, tragacanth, carrageenan, alginic acid, gum acacia, gum arabic, gellan gum, and xanthan gum; triglycerides; waxes, e.g., Compritol®, Lubritab®, and Gelucires®; lipids; fatty acids or their salts/derivatives; a mixture of polyvinyl acetate and polyvinyl pyrrolidone, e.g., Kollidon® SR; and mixtures thereof.

Suitable glidants are selected from the group comprising silica, calcium silicate, magnesium silicate, colloidal silicon dioxide, cornstarch, talc, stearic acid, magnesium stearate, calcium stearate, sodium stearyl fumarate, hydrogenated vegetable oil, and mixtures thereof.

Suitable sweeteners are selected from the group comprising saccharine or its salts such as sodium, potassium, or calcium, cyclamate or its salt, aspartame, alitame, acesulfame or its salt, stevioside, glycyrrhizin or its derivatives, sucralose, and mixtures thereof.

Suitable anti-caking agents are selected from the group comprising colloidal silicon dioxide, tribasic calcium phosphate, powdered cellulose, magnesium trisilicate, starch, and mixtures thereof.

Suitable wetting agents are selected from the group comprising anionic, cationic, nonionic, or zwitterionic surfactants, or combinations thereof. Suitable examples of wetting agents are sodium lauryl sulphate; cetrimide; polyethylene glycols; polyoxyethylene-polyoxypropylene block copolymers such as poloxamers; polyglycerin fatty acid esters such as decaglyceryl monolaurate and decaglyceryl monomyristate; sorbitan fatty acid esters such as sorbitan monostearate; polyoxyethylene sorbitan fatty acid esters such as polyoxyethylene sorbitan monooleate; polyethylene glycol fatty acid esters such as polyoxyethylene monostearate; polyoxyethylene alkyl ethers such as polyoxyethylene lauryl ether; polyoxyethylene castor oil; and mixtures thereof.

Suitable preservatives are selected from the group comprising parabens such as methyl paraben and propyl paraben; sodium benzoate; and mixtures thereof.

Suitable buffering agents are selected from the group comprising citric acid, sodium citrate, sodium phosphate, potassium citrate, acetate buffer, and mixtures thereof.

Suitable flavoring agents are selected from the group consisting of peppermint, grapefruit, orange, lime, lemon, mandarin, pineapple, strawberry, raspberry, mango, passion fruit, kiwi, apple, pear, peach, apricot, cherry, grape, banana, cranberry, blueberry, black currant, red currant, gooseberry, lingon berries, cumin, thyme, basil, camille, valerian, fennel, parsley, chamomile, tarragon, lavender, dill, bargamot, salvia, aloe vera balsam, spearmint, eucalyptus, and combinations thereof.

Suitable anti-oxidants are selected from the group comprising butylated hydroxytoluene (BHT), butylated hydroxyanisole (BHA), sodium metabisulfite, ascorbic acid, propyl gallate, thiourea, tocopherols, beta-carotene, and mixtures thereof.

Suitable chelating agents are selected from the group comprising ethylenediamine tetraacetic acid or derivatives/salts thereof, e.g., disodium edetate; dihydroxyethyl glycine; glucamine; acids, e.g., citric acid, tartaric acid, gluconic acid, and phosphoric acid; and mixtures thereof.

The ion-exchange resins such as cation- and anion-exchange matrices are well-known in the art. Few exemplary resin particles that can be used according to the invention include, but are not limited to, Dowex® resins and others made by Dow Chemical; Amberlite®, Amberlyst® and other resins made by Rohm and Haas; Indion® resins made by Ion Exchange, Ltd. (India), Diaion® resins by Mitsubishi; Type AG® and other resins by BioRad; Sephadex® and Sepharose® made by Amersham; resins by Lewatit, sold by Fluka; Toyopearl® resins by Toyo Soda; IONAC® and Whatman® resins sold by VWR; and BakerBond® resins sold by J T Baker; resins having polymer backbones comprising styrene-divinyl benzene copolymers and having pendant ammonium or tetraalkyl ammonium functional groups, available from Rohm and Haas, Philadelphia, and sold under the tradename DUOLITE™ AP143.

The cores of the present invention comprising the active ingredient can be prepared by any method known in the art, e.g., extrusion-spheronoization, wet granulation, dry granulation, hot-melt extrusion granulation, spray drying, and spray congealing. Alternatively, the active ingredient can be layered onto an inert particle to form the core. Further, the active ingredient particles can be directly coated with a release-controlling agent to form the microparticles or microcapsules. The microparticles or microcapsules can be prepared by a process of homogenization, solvent evaporation, coacervation phase separation, spray drying, spray congealing, polymer precipitation, or supercritical fluid extraction. The ion-exchange resins comprise loading a plurality of the resin particles with the active ingredient to form drug-resin cores. Methods of loading active ingredients onto the resin particles are generally known in the art.

The first chamber includes a container which is in the form of a glass or a plastic or a metallic bottle. The reservoir of the second chamber can be made of a plastic, a metal or a glass; particularly the reservoir is a plastic bottle. The reservoir of the second chamber may additionally have a slippery coating or mold polishing. This coating or polishing will help to improve the flow characteristics of the powder for suspension composition during activation.

In the dual-chamber pack suitable for incorporating powder for suspension in a volume ranging from about 0.5 cc to about 30 cc, the plunger may be inversely fitted into the plug which is subsequently screwed or snuggly fitted on to the opening of the container of the first chamber, in particularly it is screwed fitted. The overcap may be fitted screwed or snuggly into the plug, in particularly snuggly fitted. The plunger can be open at both the ends or closed at one end and open at the other end. In particular, it is open at both the ends. The plunger opened at both the ends may further increase the capacity as well as machine ability. Further, the overcap may be prefitted with the plunger. The overcap may have a tamper evident band which is removed first to start the activation.

In the dual-chamber pack suitable for incorporating for powder for suspension in a volume ranging from about 30 cc to about 500 cc, the plunger is opened at both the ends. The biphasic connector comprises of cross bridges to give the strength. The bridges can be tapered at the edges to avoid any powder deposit. Further, the reservoir can have serrations to have better grip for the end-users. The biphasic connector has a tamper-evident band on the side connected to the container of the first chamber which is removed first to start the activation process. The biphasic connector is having grooves on other side for locking with the reservoir. On this side, there would be instructions for the end-users regarding direction of the rotation such as clockwise rotation for activating the pack.

The term “tamper-evident band,” as used herein, refers to a band attached co-axially to the overcap or to the biphasic connector. The band breaks easily on pulling apart. The tamper-evident band ensures the overall integrity of the product until activation.

The plunger of the instant invention can comprise of one or more sharp projections with an essential continuous blunt area. In particular, the plunger comprise of one sharp projection with an essential continuous blunt area. Alternatively, the plunger can have a single continuous projection with a remaining continuous blunt area which can be called as a flute shaped plunger. The plunger can further have one or more grooves. The body of the plunger can be in the form of a cylinder or a funnel. The funnel shaped plunger provides additional capacity for storing high-dose active ingredients or active ingredients required for chronic administration.

The plunger used in the instant invention ensures that the breakable polymeric membrane remains attached to the plug during activation. The plug and the plunger may be made up of a material selected from the group comprising polyolefin, polyethylene, polypropylene, polyvinyl chloride, cyclic olefin polymer, cyclic olefin co-polymer, polyethylene terephthalate, polyethylene terephthalate-G, polypropylene, and polycarbonate. Particularly, the plug and the plunger are made up of polyethylene. More particularly, the plug and the plunger are made up of linear low density polyethylene (LLDPE).

The compositions of the first and second chambers of the container are separated by a polymeric breakable membrane of the plug. The plunger used in the instant invention helps to rupture the breakable polymeric membrane upon the application of pressure by a screw-based mechanism. When pressure is applied on the overcap or reservoir, the breakable polymeric membrane is ruptured by the plunger. The intact polymeric membrane remains attached to the circumference of the plug. In cases, where a bottle liner exists between the first and the second chambers, the plunger would break the bottle liner in the same manner as it ruptures the breakable polymeric membrane. The unabridged part of the bottle liner remains attached to the opening of the container. The plug with the breakable polymeric membrane prevents moisture permeation from the first chamber into the second chamber.

The material used for making the plug may also include moisture barrier additives selected from the plastic additive group comprising of monomers and co-polymers that get activated through polymerization process to form an effective organic chemical. The moisture barrier additives used in the present invention may include any material that can prevent moisture permeation. The moisture barrier additives may be present in the form of a layer inside the plug. The moisture barrier additives may be present in an amount of 0.1% to 10% w/w, in particularly, 0.5% to 5% w/w based on total weight of the material used for making plug.

The material used for making the reservoir may also include the moisture barrier additives. The barrier additives may be present in the form of a layer inside the reservoir.

The moisture permeation test was carried out on dual chamber packs with moisture barrier additives and without moisture barrier additives as per USP (37)—671 Containers Performance Testing. The moisture barrier additives used in the present invention improve the moisture barrier properties by up to 50%. In particular, the moisture barrier additives improves the moisture barrier properties by up to 30%.

The use of moisture barrier additives thus help to prevent the moisture permeation from the suspension base into the powder for suspension during storage. The active ingredient, particularly moisture-sensitive active ingredient present in the powder for suspension thus remains stable during storage.

The invention may be further illustrated by the following examples, which are for illustrative purposes only and should not be construed as limiting the scope of the invention in any way.

### EXAMPLE 1

| Ingredients | Quantity (mg/mL) |
|---|---|
| Core | |
| Metformin hydrochloride | 80.00 |
| Microcrystalline cellulose spheres | 56.00 |
| Hydroxypropylmethyl cellulose | 4.00 |
| Purified water | q.s. |
| Extended Release Coating | |
| Ethyl cellulose | 68.31 |
| Dibutyl sebacate | 1.69 |
| Acetone | q.s. |
| Purified water | q.s. |
| Total Weight of Extended | 210.00 mg |
| Release Beads | |
| Suspension base | |
| Metformin hydrochloride | 20.00 |
| Xylitol | 450.00 |
| Microcrystalline cellulose-sodium | 20.00 |
| carboxymethyl cellulose | |
| (Avicel ® CL-611) | |
| Xanthan gum | 1.50 |
| Methyl paraben | 1.80 |
| Propyl paraben | 0.20 |
| Strawberry flavor | 2.00 |
| Sucralose | 0.50 |
| Colloidal silicon dioxide | 3.50 |
| Purified water | 472.00 mg |

### Procedure:

1. Metformin hydrochloride and hydroxypropylmethyl cellulose were dissolved in purified water.
2. Microcrystalline cellulose spheres were coated with the solution of step 1.
3. Ethyl cellulose and dibutyl sebacate were dispersed in a mixture of acetone and purified water.
4. The beads of step 2 were coated with the coating dispersion of step 3 and dried to form a powder for suspension.
5. Purified water was heated to dissolve methyl paraben and propyl paraben.
6. Metformin hydrochloride, xylitol, microcrystalline cellulose-sodium carboxymethyl cellulose, xanthan gum, strawberry flavor, sucralose, and colloidal silicon dioxide were mixed in the solution of step 5 to form a suspension base.
7. The powder for suspension of step 4 was prefilled in a plunger of a second chamber of a dual-chamber pack.
8. The suspension base of step 6 was prefilled in a container of a first chamber of a dual-chamber pack.
9. The two chambers were assembled and the pack was activated to form the extended release suspension composition when required.

### In-Vitro Studies

The extended release suspension composition prepared as per Example 1 (for a dose equivalent to 750 mg of metformin hydrochloride) was stored at room temperature for 120 days. The in-vitro dissolution was determined at 0, 45, 90, and 120 days using USP type II apparatus at 100 rpm, in 1000 mL of phosphate buffer with pH 6.8 at 37° C. The results of the release studies are represented in Table 1.

TABLE 1
Percentage (%) of the In-Vitro Metformin
Release in USP Type II Apparatus
(Media: Phosphate Buffer,
pH 6.8, 1000 mL, and 100 rpm)

| | Number of Days | | | |
|---|---|---|---|---|
| | 0 | 45 | 90 | 120 |
| Time (hours) | Percentage of Metformin Release | | | |
| 0.5 | 20 | 21 | 20 | 21 |
| 1 | 27 | 25 | 27 | 25 |
| 2 | 55 | 52 | 55 | 52 |
| 3 | 74 | 72 | 74 | 72 |
| 4 | 83 | 81 | 83 | 81 |
| 5 | 85 | 86 | 85 | 86 |
| 6 | 87 | 90 | 87 | 90 |
| 8 | 91 | 94 | 91 | 94 |
| 10 | 93 | 96 | 93 | 96 |
| 12 | 94 | 97 | 94 | 97 |

From the above in-vitro release data, it is evident that the extended release suspension composition prepared according to Example 1 provides the substantially similar in-vitro metformin release for 120 days.

The dual-chamber pack was kept for 1 month at accelerated conditions i.e., 40° C/75% R.H. After 1 month, the pack was activated to form an extended release liquid composition which was kept for 120 days at room temperature. The in-vitro dissolution was determined at 0, 45, 90, and 120 days using USP type II apparatus at 100 rpm, in 1000 mL of phosphate buffer with pH 6.8 at 37° C. The results of the release studies are represented in Table 2.

TABLE 2
Percentage (%) of the In-Vitro Metformin Release
in USP Type II Apparatus (Media: Phosphate Buffer,
pH 6.8, 1000 mL, and 100 rpm)

| | Number of Days | | | |
|---|---|---|---|---|
| Time (hours) | 0 | 45 | 90 | 120 |
| Release | Percentage of Metformin | | | |
| 0.5 | 21 | 21 | 21 | 20 |
| 1 | 27 | 25 | 26 | 26 |
| 2 | 56 | 55 | 52 | 54 |
| 3 | 74 | 74 | 76 | 72 |
| 4 | 83 | 81 | 82 | 81 |
| 10 | 96 | 96 | 97 | 94 |

The dual-chamber pack was kept for 3 months at accelerated conditions i.e., 40° C/75% R.H. After 3 months, the pack was activated to form an extended release liquid composition which was kept for 45 days at room temperature. The in-vitro dissolution was determined at 0 and 45 days using USP type II apparatus at 100 rpm, in 1000 mL of phosphate buffer with pH 6.8 at 37° C. The results of the release studies are represented in Table 3.

TABLE 3
Percentage (%) of the In-Vitro Metformin
Release in USP Type II Apparatus (Media:
Phosphate Buffer, pH 6.8, 1000 mL, and 100 rpm)

| | Number of Days | |
|---|---|---|
| Time | 0 | 45 |
| (hours) | Percentage of Metformin Release | |
| 0.5 | 21 | 21 |
| 1 | 26 | 25 |
| 2 | 55 | 53 |
| 3 | 75 | 72 |
| 4 | 80 | 80 |
| 10 | 95 | 92 |

From the above data, it is clear that the powder for suspension and suspension base stored in the dual-chamber pack of the instant invention at accelerated conditions for 1 month and 3 months, upon activation of the pack forms extended release suspension compositions which when stored for 120 days and 45 days respectively at room temperature provides substantially similar in-vitro metformin release.

### Stability Data

The related substances for the extended release suspension composition prepared as per Example 1 were determined at 0 day and after storage at room temperature for 45 and 120 days. The powder for suspension and suspension base was stored in the dual-chamber pack for one month and for three months at 40° C/75% R.H. After one month or three months, the pack was activated to form extended release suspension compositions and then related substances were determined at 0 day and after storage at room temperature for 45 days and 120 days.

The assay of metformin was determined by HPLC method. The results are shown in Table 4.

TABLE 4
Stability Data for Metformin

| | | | | 1 month | | | 3 month | |
|---|---|---|---|---|---|---|---|---|
| | | | | (40° C./75% | | | (40° C./75% | |
| Related | Initial | | | R.H) | | | R.H) | |
| Substances | | 45 | 120 | | 45 | 120 | | 45 |
| (% w/w) | 0 day | days | days | 0 day | days | days | 0 day | days |
| Cyano- | BLQ | 0.001 | 0.00072 | 0.001 | 0.001 | 0.001 | 0.001 | 0.001 |
| guainidine | | | | | | | | |
| Highest | 0.05 | 0.05 | 0.04 | 0.05 | 0.04 | 0.04 | 0.05 | 0.04 |
| unknown | | | | | | | | |
| impurity | | | | | | | | |
| Total | 0.05 | 0.05 | 0.04 | 0.05 | 0.04 | 0.04 | 0.09 | 0.04 |
| impurities | | | | | | | | |

*BLQ: Below limit of Quantification

It is evident from the above data that the extended release suspension composition prepared as per Example 1 remains stable even after storing at accelerated conditions for 3 months using the dual-chamber pack.

### EXAMPLE 2

| Ingredients | Quantity (mg/mL) |
|---|---|
| Core | |
| Metformin hydrochloride | 80.00 |
| Microcrystalline cellulose spheres | 56.00 |
| Hydroxypropylmethyl cellulose | 4.00 |
| Purified water | q.s. |
| Extended Release Coating | |
| Ethyl cellulose | 61.48 |
| Dibutyl sebacate | 1.52 |
| Acetone | q.s. |
| Purified water | q.s. |
| Total Weight of Extended Release Beads | 203.00 mg |
| Metformin hydrochloride | 20.00 |
| Xylitol | 450.00 |
| Microcrystalline cellulose-sodium | 20.00 |
| carboxymethyl cellulose | |
| (Avicel ® CL-611) | |
| Xanthan gum | 1.50 |
| Strawberry flavor | 2.00 |
| Sucralose | 0.50 |
| Colloidal silicon dioxide | 3.50 |
| Vehicle | |
| Purified water | q.s. to 1 mL |

### Procedure:

1. Metformin hydrochloride and hydroxypropylmethyl cellulose were dissolved in purified water.
2. Microcrystalline cellulose spheres were coated with the solution of step 1.
3. Ethyl cellulose and dibutyl sebacate were dispersed in a mixture of acetone and purified water.
4. The beads of step 2 were coated with the coating dispersion of step 3.
5. Metformin hydrochloride, xylitol, microcrystalline cellulose-sodium carboxymethyl cellulose, xanthan gum, strawberry flavor, sucralose, and colloidal silicon dioxide were mixed.
6. The coated beads of step 4 were mixed with the mixture of step 5 to form a powder for suspension.

The powder for suspension may be stored in plunger or reservoir (depending upon the volume) of the second chamber and the vehicle (purified water) may be stored in the container of the first chamber. The two chambers after assembling may be activated to form the extended release suspension composition when required.

### In-Vitro Studies

The extended release suspension composition prepared as per Example 2 was stored at room temperature for 30 days. The in-vitro dissolution was determined at 0 and 30 days using USP type II apparatus at 100 rpm, in 1000 mL of phosphate buffer with pH 6.8 at 37° C. The results of the release studies are represented in Table 5.

TABLE 5
Percentage (%) of the In-Vitro Metformin Release in USP
Type II Apparatus (Media: Phosphate Buffer,
pH 6.8, 1000 mL, and 100 rpm)

| | Number of Days | |
|---|---|---|
| | 0 | 30 |
| Time (hours) | Percentage of Metformin Release | |
| 0.5 | 20 | 22 |
| 1 | 27 | 28 |
| 2 | 59 | 64 |
| 3 | 77 | 80 |
| 4 | 84 | 89 |
| 5 | 88 | 93 |
| 6 | 92 | 95 |
| 8 | 95 | 99 |
| 10 | 97 | 101 |
| 12 | 98 | 103 |

From the above in-vitro release data, it is evident that the extended release suspension composition prepared according to Example 2 provides the substantially similar in-vitro metformin release for 30 days.

### Osmolality Measurement of the Extended Release Suspension

The metformin extended release powder prepared according to the Example 2 (till step 6) was reconstituted with required amount of purified water. This suspension was shaken manually for at least 20 minutes. This suspension was then filtered and diluted with purified water and the osmolality was measured using Osmomat 030-D.

The osmolality of the suspension base was found to be 4.112 osmol/kg of the suspension base on day 0.

The osmolality of the suspension base was found to be 4.328 osmol/kg of the suspension base on day 7.

It is evident from the above data that the osmolality of the suspension base of the extended release suspension composition as per Example 2 remains equivalent for seven days.

### Osmolality Measurement of the External Phase

The metformin hydrochloride, xylitol, microcrystalline cellulose-sodium carboxymethyl cellulose, xanthan gum, strawberry flavor, sucralose, and colloidal silicon dioxide were mixed as per step 5 of Example 2. This mixture was reconstituted with required amount of purified water. This suspension was then filtered and diluted with purified water, and the osmolality was measured using Osmomat 030-D.

The osmolality of the suspension base i.e., external phase was found to be 4.204 osmol/kg of the suspension base.

### Osmolality Measurement of the Internal Phase

Various solutions having various concentrations of osmogent (sodium chloride) were prepared as per Examples 2A-2F. The osmolalities of these solutions were measured using Osmomat 030-D.

| | Example | Example | Example | Example | Example | Example |
|---|---|---|---|---|---|---|
| Ingredient | 2A | 2B | 2C | 2D | 2E | 2F |
| Sodium Chloride | 30.00 | 60.00 | 120.00 | 180.00 | 240.00 | 300.00 |
| (mg) | | | | | | |
| Purified water | q.s. to 1 mL | q.s. to 1 mL | q.s. to 1 mL | q.s. to | q.s. to 1 mL | q.s. to 1 mL |
| | | | | 7.5 mL | | |
| Osmolality | 0.910 | 1.787 | 3.574* | 5.361* | 7.148* | 8.935* |
| (osmol/kg) | | | | | | |

*Extrapolated using values of dilute solutions

The coated beads of step 4 were dispersed in different solutions as per Examples 2A-2F. These solutions were kept for seven days at room temperature. After seven days, each solution was analyzed by HPLC for metformin content. The results are represented in following Table 5.

TABLE 5
Effect of Osmolality on Metformin Leaching

| | Osmolality (osmol/kg) | Metformin |
|---|---|---|
| Example | of the solution | Content (%) |
| 2A | 0.910 | 67.3 |
| 2B | 1.787 | 30.3 |
| 2C | 3.574* | 2.9 |
| 2D | 5.361* | 1.8 |
| 2E | 7.148* | 1.7 |
| 2F | 8.935* | 1.0 |

*Extrapolated using values of dilute solutions

From the above data, it is evident that the leaching of metformin from the coated beads into the solution was decreasing as the osmolality of the solution was increasing from Examples 2A-2F. The leaching is found to be significantly reduced from Example 2C onwards. The osmolality of Example 2C i.e., 3.574 is considered as osmolality of the internal phase.

### Osmolality Ratio 1.176

We claim:

1. A dual-chamber pack comprising;
(a) a first chamber prefilled with a suspension base; and
(b) a second chamber prefilled with a powder for suspension comprising an active ingredient;
wherein upon activation of the dual-chamber pack, the contents of both the chambers are mixed to form an extended release suspension composition which is characterized by having no substantial change in the in-vitro dissolution release profile of the active ingredient upon storage for at least seven days.

2-3. (canceled)

4. The dual-chamber pack of claim 1, wherein the first chamber comprises of a container and the second chamber comprises of a reservoir, a biphasic connector, a plunger, and a plug with a breakable polymeric membrane.

5. The dual-chamber pack of claim 4, wherein the reservoir is prefilled with the powder for suspension in a volume greater than about 30 cc.

6. The dual-chamber pack of claim 5, wherein the reservoir is prefilled with the powder for suspension in a volume ranging from about 30 cc to about 500 cc.

7. The dual-chamber pack of claim 4, wherein the biphasic connector of the second chamber connects the reservoir of the second chamber to the container of the first chamber.

8-10. (canceled)

11. The dual-chamber pack of claim 1, wherein the extended release suspension composition is a stable composition.

12-13. (canceled)

14. A dual-chamber pack comprising:
(a) a first chamber in the form of a container (8) prefilled with a suspension base provided with an opening (7) at an upper end;
(b) a second chamber comprising:
(i) a reservoir (1) adapted to fit into a plunger (2) prefilled with a powder for suspension; the plunger (2) is further adapted to fit into a plug (3) having a top flat surface,
(ii) the plug (3), with a breakable polymeric membrane (4), adapted to fit into the biphasic connector (5) optionally having a tamper evident band (6) which is further connected from the lower end to the opening (7) of the container (8);
wherein the reservoir (1) at the top of the second chamber has a means to exert pressure onto the plunger (2) so as to partially rupture the breakable polymeric membrane (4) of the plug and deliver the powder for suspension into the suspension base of the container (8); the second chamber is replaced with a cap (9), and wherein the powder for suspension is mixed with the suspension base to form an extended release suspension composition which is characterized by having no substantial change in the in-vitro dissolution release profile for at least seven days upon storage.

15. The dual-chamber pack of claim 14, wherein the reservoir is prefilled with the powder for suspension in a volume greater than about 30 cc.

16. The dual-chamber pack of claim 15, wherein the reservoir is prefilled with the powder for suspension in a volume ranging from about 30 cc to about 500 cc.

17. The dual-chamber pack of claim 1, wherein the powder for suspension comprises of extended release coated cores of the active ingredient, optionally admixed with one or more pharmaceutically acceptable excipients.

18. The dual-chamber pack of claim 17, wherein the extended release coated cores comprise a core comprising the active ingredient and a coating layer over said core comprising one or more release-controlling agents.

19. The dual-chamber pack of claim 1, wherein the active ingredient is selected from the group consisting of valacyclovir, metformin, azithromycin, cloxacillin, clarithromycin, erythromycin, amoxicillin alone or in combination with clavulanic acid, cefdinir, cefuroxime axetil, cefixime, cefadroxil, cefpodoxime, cefaclor, cefprozil, fluconazole, voriconazole, acarbose, miglitol, voglibose, repaglinide, nateglinide, glibenclamide, glimepride, glipizide, gliclazide, chloropropamide, tolbutamide, phenformin, alogliptin, sitagliptin, linagliptin, saxagliptin, rosiglitazone, pioglitazone, troglitazone, faraglitazar, englitazone, darglitazone, isaglitazone, zorglitazone, liraglutide, muraglitazar, peliglitazar, tesaglitazar, canagliflozin, dapagliflozin, remogliflozin, sergliflozin, verapamil, albuterol, salmeterol, acebutolol, sotalol, penicillamine, norfloxacin, ciprofloxacin, ofloxacin, levofloxacin, moxifloxacin, trovafloxacin, gatifloxacin, tetracycline, demeclocycline hydrochloride, losartan, irbesartan, eprosartan, valsartan, diltiazem, isosorbide mononitrate, ranolazine, propafenone, hydroxyurea, hydrocodone, delavirdine, pentosan polysulfate, abacavir, amantadine, acyclovir, ganciclovir, valganciclovir, saquinavir, indinavir, nelfinavir, lamivudine, didanosine, zidovudine, nabumetone, celecoxib, mefenamic acid, naproxen, propoxyphene, cimetidine, ranitidine, albendazole, mebendazole, thiobendazole, pyrazinamide, praziquantel, chlorpromazine, sumatriptan, bupropion, aminobenzoate, pyridostigmine bromide, potassium chloride, niacin, tocainide, quetiapine, fexofenadine, sertraline, chlorpheniramine, rifampin, methenamine, nefazodone, modafinil, metaxalone, morphine, sevelamer, lithium carbonate, flecainide acetate, simethicone, methyldopa, chlorthiazide, metyrosine, procainamide, entacapone, metoprolol, propanolol hydrochloride, chlorzoxazone, tolmetin, tramadol, bepridil, phenytoin, gabapentin, terbinafine, atorvastatin, doxepine, rifabutin, mesalamine, etidronate, nitrofurantoin, choline magnesium trisalicylate, theophylline, nizatidine, methocarbamol, mycophenolate mofetil, tolcapone, ticlopidine, capecitabine, orlistat, colsevelam, meperidine, hydroxychloroquine, guaifenesin, guanfacine, amiodarone, quinidine, atomoxetine, felbamate, pseudoephedrine, carisoprodol, venlafaxine, etodolac, chondroitin, lansoprazole, pantoprazole, esomeprazole, dexlansoprazole, dexmethylphenidate, methylphenidate, sodium oxybate, valproic acid or its salts, divalproex, topiramate, carbamazepine, oxcarbazepine, isotretinoin, oseltamivir, cholestyramine, nystatin, and a combination of artemether and lumefantrine.

20. The dual-chamber pack of claim 18, wherein the release-controlling agents are selected from the group consisting of a pH-dependent release-controlling agent, a pH-independent release-controlling agent, and mixtures thereof.

21. The dual-chamber pack of claim 20, wherein the pH-dependent release-controlling agent is selected form the group consisting of acrylic copolymers; cellulose acetate phthalate; cellulose acetate succinates; hydroxyalkyl cellulose phthalates; hydroxyalkyl cellulose acetate succinates; vinyl acetate phthalates; vinyl acetate succinate; cellulose acetate trimelliate; polyvinyl derivatives; zein; shellac; and mixtures thereof.

22. The dual-chamber pack of claim 20, wherein the pH-independent release-controlling agent is selected from the group consisting of cellulosic polymers; acrylic copolymers; cellulose acetate; polyethylene derivatives; polyvinyl alcohol; polyvinyl acetate; gums; triglycerides; waxes; a mixture of polyvinyl acetate and polyvinyl pyrrolidone; and mixtures thereof.

23. The dual-chamber pack of claim 1, wherein the suspension base comprises one or more suspending agents, one or more osmogents, and a pharmaceutically acceptable vehicle.

24. The dual-chamber pack of claim 17, wherein the pharmaceutically acceptable excipients are selected from the group consisting of glidants, sweeteners, suspending agents, anti-caking agents, wetting agents, preservatives, buffering agents, flavoring agents, anti-oxidants, chelating agents, and combinations thereof.

25. The dual-chamber pack of claim 4, wherein the plunger ensures the breakable polymeric membrane remains attached to the plug during activation.

26. The dual-chamber pack of claim 4, wherein the plunger comprises one or more sharp projections with an essential continuous blunt area.

27. The dual-chamber pack of claim 4, wherein the plug includes one or more moisture barrier additives.

28. The dual-chamber pack of claim 21, wherein the acrylic copolymer is methacrylic acid or methyl methacrylate copolymers; wherein the methyl methacrylate copolymer is Eudragit® L 100 and Eudragit® S 100, methacrylic acid or ethyl acrylate copolymers, or methacrylic acid and ethyl acrylate copolymers; wherein the ethyl acrylate copolymer is Eudragit® L 100-55 and Eudragit® L 30 D-55, dimethylaminoethyl methacrylate and butyl methacrylate, or methyl methacrylate copolymers; wherein the methyl methacrylate copolymer is Eudragit® E 100, Eudragit® E PO, methyl acrylate and methacrylic acid and octyl acrylate copolymers, styrene and acrylic acid copolymers, butyl acrylate and styrene and acrylic acid copolymers, or ethylacrylate-methacrylic acid copolymers; wherein the hydroxyalkyl cellulose phthalate is hydroxypropylmethyl cellulose phthalate; wherein the hydroxyalkyl cellulose acetate succinates is hydroxypropylmethyl cellulose acetate succinate; wherein the polyvinyl derivatives is polyvinyl acetate phthalate, polyvinyl alcohol phthalate, polyvinyl butylate phthalate, or polyvinyl acetoacetal phthalate.

29. The dual-chamber pack of claim 22, wherein the acrylic copolymers is methacrylic acid copolymers, wherein the methacrylic acid copolymers are Eudragit® RS, Eudragit® RL, orEudragit® NE 30 D; wherein the polyethylene derivatives are polyethylene glycol or polyethylene oxide; wherein the gum is guar gum, locust bean gum, tragacanth, carrageenan, alginic acid, gum acacia, gum arabic, gellan gum, or xanthan gum; wherein the wax is Compritol®, Lubritab®, or Gelucires®; wherein the polyvinyl pyrrolidone is Kollidon® SR.
